(19) United States
(12) Patent Application Publication
(10) Pub. No.: US 2020/0397711 A1
(43) Pub. Date: Dec. 24, 2020

(54) MICROPARTICLE COMPOSITIONS FOR TREATMENT OF INFECTION OR DISEASE, METHODS OF MAKING THE SAME, AND METHODS OF TREATING SUBJECTS WITH MICROPARTICLE COMPOSITIONS

(71) Applicant: Gregory Brian LEE, Frederick, MD (US)

(72) Inventor: Gregory Brian LEE, Frederick, MD (US)

(21) Appl. No.: 16/908,586

(22) Filed: Jun. 22, 2020

Related U.S. Application Data
(60) Provisional application No. 62/865,205, filed on Jun. 22, 2019.

(51) Int. Cl. A61K 9/51; A61K 9/127; A61K 9/107

(52) U.S. Cl. CPC A61K 9/51; A61K 45/06; A61K 9/1075; A61K 9/127

(57) ABSTRACT

Provided are microparticle compositions, methods of making the compositions, and methods of treating a subject with the present microparticle compositions by administering an effective amount of the microparticle composition for treating an infection or disease, to a subject in need thereof. The present microparticle compositions may include microparticles of essential oils, herbs, and/or supplements. By way of non-limiting example, the microparticle compositions may be particularly effective in the treatment of a Lyme tick borne illness related infection or disease, or a coronavirus, such as COVID-19. Also included are kits that include one or more of the present microparticle compositions and/or one or more components of the present compositions, and optionally one or more components, such as a device to aid in the delivery of the composition and/or instructions for administration.

### FIELD

The present disclosure relates generally to microparticle compositions for treatment of infection or disease, methods of making such compositions, and methods of treating a subject with the present microparticle therapy compositions. The present disclosure also includes kits that include one or more of the present microparticle compositions and/or one or more components of the present compositions, and optionally one or more components, such as a device to aid in the delivery of the composition and/or instructions for administration.

### BACKGROUND

According to current estimates, the prevalence of Lyme disease infections is estimated to range from a low of 329,000 cases by the Centers for Disease Control (CDC) to a high of over 2,000,000 by 2020.

Current standards of treatment for tick-borne infections employ antibiotic medications, which may drive the creation of debilitating or deadly infections. Unfortunately, these medications have been shown to produce drug-resistant mutant germs that can survive despite being treated with multiple rounds of antimicrobial drugs. Drug resistant forms of tick-borne infections including: Borrelia, Babesia, Bartonella, Ehrlichia, and Anaplasmosis have been identified. Medical providers are deeply concerned that as more and more patients receive antibiotic treatment these infections will eventually become increasingly resistant to their limited number of antimicrobial drugs.

Drug resistance is a global concern, with drug resistant infections are rising worldwide. According to the World Health Organization (WHO), approximately 700,000 deaths worldwide every year are due to drug-resistant infections. These and other factors have led the WHO to predict a dramatic increase in the severity of drug resistant infections. Deaths from drug-resistant infections are estimated to rise to 10 million worldwide by 2050.

The wide-spread excess use of antibiotics in livestock for the purpose of increasing animal weight, not for treating infections, is believed to be a major driver of mutations of new multidrug resistant superbugs. Drug resistant tick-borne infections may arise in these same livestock.

Tick infections are also ripe for developing multidrug resistant superbugs in these same livestock that get infected from tick bites and are over treated with antibiotics. These superbugs may be spread through the environment by blood sucking ticks that feed on these superbug infected livestock and crawl off to infect humans, pets, and other animals. The superbug microbes have been found in the manure and water runoff of these large-scale factory farms. These highly resistant germs have also been found in underground well water near these farms where conditions are highly favorable for their growth. These superbugs may also be spread when contaminated water is used for drinking or watering crops. Given that the FDA has left it up to pharmaceutical companies to regulate antibiotic use for increasing livestock weight, it is likely that we will see increased drug resistance in tick borne infections.

Also of concern, multiple tick-borne infections including Powassan fever, Heartland virus, Colorado tick fever, Tick-borne encephalitis, Bourbon virus, and southern tick-associated rash illness (STARI) are lacking any effective medications according to the CDC.

### SUMMARY

Non-antibiotic therapy compositions would be advantageous for treatment of tick-borne illnesses, as well as for other illnesses.

The present disclosure relates generally to microparticle compositions or microparticle therapy compositions, kits including such compositions and/or components thereof, methods of making such compositions, and methods of treating a subject with the present microparticle compositions by administering an effective amount of a composition to the subject, to treat one or more infections or disease in the subject.

By way of non-limiting example, the present microparticle therapy compositions may include a liposome, nanoparticle, nanostructured lipid carriers (NLCs), or nano-emulsion composition that includes one or more of the following: GRAS (generally recognized as safe) essential oils, herbs, and supplements, including those presently available, as well as those that maybe identified and become available in the future. According to non-limiting examples, the present compositions may be formulated in amount and type of ingredients, based on the present inventor's discoveries, to achieve the desired advantageous results discovered by the present inventor.

Non-limiting examples of infections that may be treated using the present compositions and methods may include bacterial, viral and/or fungal infections. By way of non-limiting example, the invention may include one or more Lyme tick borne infections or diseases, or a coronavirus infection, such as COVID-19.

Methods of treatment may include treating a subject/patient having an infection with one or more of the present microparticle compositions, by administering an effective amount of the composition(s) to a subject/patient in need of such treatment.

Also included herein are kits that include one or more of the present microparticle therapy compositions or one or more components of said compositions and optionally one or more components, such as a device to aid in the delivery of the composition and/or instructions for making the composition or administration to a patient in need thereof.

Methods of making the present compositions are also provided herein.

### DETAILED DESCRIPTION

The present inventor has discovered microparticle therapy compositions that include essential oils, such as Generally Recognized as Safe (GRAS) essential oils, herbs, and/or supplements, which advantageously kill infections, such as drug-resistant, drug-persisters, biofilms, and cyst forms of infections. Essential oils, herbs and supplements are made up of several compounds which makes it much more difficult for pathogens to develop resistance to. The present formulations and compositions are believed to be superior in the treatment and/or prevention of, inter alia, a coronavirus infection or tick-borne infections and disease, such as drug-resistant forms. The present formulations and compositions are also believed to effective in treatment and/or prevention of other infections and/or disease, including, but not limited to a coronavirus, such as COVID-19.

The aspects, advantages and/or other features of example embodiments of the present disclosure will become apparent to those skilled in the art, in view of the present detailed description. It should be apparent to those skilled in the art that the described embodiments of the present disclosure provided herein are merely exemplary and illustrative and not limiting. Numerous embodiments of modifications thereof are contemplated as falling within the scope of the present disclosure and equivalents thereto. Unless otherwise noted, technical terms are used according to conventional usage. Any publications mentioned in this specification are indicative of the level of those skilled in the art to which the invention pertains and are incorporated by reference in their entirety. For example, certain of the references cited herein (including Low, W. L., C. Martin, D. J. Hill, and M. A. Kenward. “Antimicrobial Efficacy of Liposome-Encapsulated Silver Ions and Tea Tree Oil against Pseudomonas aeruginosa, Staphylococcus aureus and Candida albicans.” Letters in Applied Microbiology 57, no. 1 (July 2013): 33-39. https://doi.org/10.1111/lam.12082 and Cui, Haiying, Wei Li, Changzhu Li, Saritporn Vittayapadung, and Lin Lin. “Liposome Containing Cinnamon Oil with Antibacterial Activity against Methicillin-Resistant Staphylococcus aureus Biofilm.” Biofouling 32, no. 2 (2016): 215-25. https://doi.org/10.1080/08927014.2015.1134516, for example. These references include examples showing the process of forming essential oil microparticles and testing such microparticle remedies against pathogens.

In describing example embodiments, specific terminology is employed for the sake of clarity. However, the embodiments are not intended to be limited to this specific terminology.

As used herein, “a” or “an” may mean one or more. As used herein, “another” may mean at least a second or more. Furthermore, unless otherwise required by context, singular terms include pluralities and plural terms include the singular.

As used herein, the terms “composition”, “therapeutic composition”, and “formulation” may be used interchangeably and refer to a combination of elements that is presented together for a given purpose. Such terms are well known to those of ordinary skill in the art.

As used herein, “an effective amount” refers to an amount of one or more active ingredients in a composition, or an amount of the overall composition that is effective in attaining desired results, the purpose for which the constituent or composition is provided. Therefore, an effective amount of a composition would be an amount suitable for achieving the desired effects in a subject, such as a mammal (e.g., human) to which the present composition is administered. Desired results or a desired effect may include for example, providing prevention or lessening the likelihood of being infected with an infection or of contracting a disease; or the desired effect may lessen the infection or disease or symptoms thereof; or the desired effect may include lessening length of duration of the infection or disease or symptoms thereof.

As indicated above, the present disclosure relates generally to microparticle therapy compositions, kits including such compositions or components thereof, methods of making such compositions, and methods of treating a subject with the present microparticle compositions by administering an effective amount of a composition to a subject in need thereof to treat one or more infections. The microparticles may be in liposome form. Other microparticles, including certain essential oil microparticles may be in the form of emulsions, nano-emulsions, nanoparticles, nanostructured lipid carriers, etc., and may not necessarily be for example, in liposome or other forms as discussed further herein.

By way of non-limiting example, the present microparticle therapies, or therapy compositions, may include a liposome, nanoparticle, nanostructured lipid carriers (NLCs), or nano-emulsion composition that includes one or more of the following: GRAS (generally recognized as safe) essential oils, herbs, and supplements, including those presently available, as well as those that maybe identified and become available in the future. According to non-limiting example embodiments, the present compositions may be formulated in amount and type of ingredients, based on the present inventor's discoveries, to achieve the desired advantageous results discovered by the present inventor. The Food and Drug Administration (FDA) has classified hundreds of essential oils, herbs, and supplements as Generally Recognized as Safe (GRAS) for internal consumption. Accordingly, those skilled in the art would be able to determine which essential oils, herbs, and supplements are GRAS now and at a future time, as additional essential oils, herbs and supplements may be determined to be GRAS for internal consumption in the future.

Essential oils that may be used in accordance with the present invention include those mentioned herein, as well as other essential oils that may be used as a therapy or treatment for an illness or disease when modified to be in microparticle form. Non-limiting examples of essential oils that may be used in accordance with embodiments of the present invention include the following:

ESSENTIAL OIL, SYNONYMS (botanical name(s))
African bluegrass (Cymbopogon validus)
Agarwood (Aquilaria agallocha Roxb., Agallochum malaccense (Lamk) Kuntze, Aquilariella malaccensis)
Ajowan (Trachyspermum ammi L., Carum copticum L.)
Almond (bitter, FFPA) Botanical name: Prunus dulcis (Mill.) var. amara, Prunus communis L. var. amara,
Prunus amygdalus, Batsch. var. amara, Amydalus communis L. var. amara, Amygdalus dulcis
Ambrette seed, musk seed (Abelmoschus moschatus Medik., Hibiscus abelmoschus L.)
Amyris (Amyris balsamifera L.)
Angelica root (Angelica archangelica L., Angelica glauca Edgew.)
Angelica seed (Angelica archangelica L., Angelica glauca Edgew.)
Anise seed (Pimpinella anisum L.)
Anise star (Illicium verum J. D. Hook.)
Artemisia annua (Artemisia annua L.)
Asafoetida (Ferula asa-foetida L.)
Atractylis, Cang-zhu, atractylodes (Atractylodes lancea (Thunb.) DC)
Bakul, Bakula, vakul, vakul (Mimusops elengi L.)
Balsamite, Costmary, alecost (Chrysanthemum balsamita L., Balsamita major Desf., Pyrethrum majus Desf.)
Balsam poplar, tacamahac, black poplar (Populus balsamifera L.)
Basil (Ocimum basilicum L.)
Basil holy, Sacred basil, tulsi (Ocimum tenuiflorum L., Ocimum sanctum L.)
Basil Madagascan, Romba, ramy (Ocimum gratissimum L., Ocimum viride Willd.)
Bay West Indian, (Pimenta racemosa var. racemosa (Miller) J. Moore, Pimenta acris Wight)
Benzoin, Gum Benjamin (Styrax benzoin Dryand, Styrax paralleloneurus Perkins Styrax tonkinensis Pierre)
Bergamot FCF, Rectified bergamot, furanocoumarin-free bergamot (Citrus bergamia Risso & Poit.,
Citrus aurantium subsp. bergamia Risso & Poit.)
Birch sweet, Black birch, cherry birch, mahogany birch, southern birch (Betula lenta L.)
Black seed, Black cumin, black caraway, Roman coriander, fennel flower, nutmeg flower (Nigella sativa L.)
Cajuput, Cajeput, cajaput, river tea tree, swamp tea tree, weeping tea tree, punk tree (Melaleuca cajuputi
Powell, Melaleuca leucadendron var. cajuputi Roxb.)
Calamus diploid form, North American calamus, sweet flag (Acorus calamus L. var. americanus Raf. Wulff)
Camphor white, Hon-sho true camphor (Cinnamomum camphora L.)
Cananga (Cananga odorata Lam. J. D. Hook and T. Thompson f. macrophylla Koolhaas, Canangium odoratum
f. macrophylla)
Cangerana (Cabralea cangerana Saldanha, Cabralea canjerana Saldanha)
Caraway (Carum carvi L.)
Cardamon (Elettaria cardamomum L.)
Carrot seed (Daucus carota L. subsp. sativus Hoffm.)
Cascarilla, sweet-wood (Croton eluteria L. Sw. Bahamas, Croton reflexifolius Homb., Bonpl. & Kunth El Salvador)
Cassia, Chinese or false cinnamon (Cinnamomum cassia Blume, Cinnamomum aromaticum Nees)
Cedarwood Atlas, Atlantic cedar, Moroccan cedar (Cedrus atlantica G. Manetti)
Cedarwood Himalayan, Deodar cedarwood (Cedrus deodara Roxb. Ex D. Don)
Cedarwood Texan, Mexican cedar, Mexican juniper, mountain cedar, rock cedar (Juniperus ashei
Buchholz, Botanical synonym: Juniperus mexicana Spreng.)
Cedarwood Virginian, Red cedar, Eastern red cedar (Juniperus virginiana L.)
Celery leaf (Apium graveolens L.)
Celery seed (Apium graveolens L.)
Chamomile blue, German chamomile, Hungarian chamomile, sweet false chamomile (Matricaria recutita L.,
Chamomilla recutita L. Rauschert, Matricaria chamomilla L.)
Chamomile Roman, (Chamaemelum nobile L. All., Anthemis nobilis L.)
Champaca orange, Golden champa, champak (Michelia champaca L.)
Chaste tree, Monk's pepper (Vitex agnus castus L.)
Cinnamon bark (Cinnamomum verum J. Presl., Cinnamomum zeylanicum Blume)
Cinnamon leaf (Cinnamomum verum J. Presl., Cinnamomum zeylanicum Blume)
Cistus (Cistus ladanifer L., Cistus ladaniferus Curtis)
Citronella (Cymbopogon nardus L. synonym: Andropogon nardus L., Sri Lanka type citronella;
Cymbopogon winterianus Jowitt Java type citronella)
Clary sage, Muscatel sage (Salvia sclarea L.)
Clove bud (Syzygium aromaticum L. Merill et L. M. Perry, Eugenia caryophyllata Thunb., Eugenia aromatica L.)
Clove leaf (Syzygium aromaticum (L.) Merill et L. M. Perry, Eugenia caryophyllata Thunb., Eugenia aromatica L.)
Clove stem (Syzygium aromaticum (L.) Merill et L. M. Perry, Eugenia caryophyllata Thunb., Eugenia aromatica L.)
Copaiba (Copaifera langsdorfii Desf., Copaifera officinalis Jacq. L.)
Coriander leaf, Cilantro (Coriandrum sativum L.)
Coriander seed, Cilantro (Coriandrum sativum L.)
Cubeb, Java pepper, tailed pepper, false pepper (Piper cubeba L.)
Cumin (Cuminum cyminum L.)
Cypress, Italian cypress, Mediterranean cypress (Cupressus sempervirens L.)
Davana (Artemisia pallens Wall ex DC)
Dill seed European (Anethum graveolens L.)
Elemi (Canarium luzonicum Blume A. Gray, Canarium vulgare Leenh., Canarium commune auct.)
Eucalyptus (Eucalyptus camaldulensis Dehnh., Eucalyptus globulus Labill. blue gum, Tasmanian blue
gum; Eucalyptus maidenii F. Muell. Maiden's gum; Eucalyptus plenissima Gardner Brooker,
Eucalyptus kochii subsp. plenissima Gardner; Eucalyptus polybractea R. T. Baker blue-leaved mallee;
Eucalyptus radiata Sieber ex DC, Eucalyptus Australiana R. T. Baker, Eucalyptus phellandra R. T. Baker,
narrow-leaved peppermint, grey peppermint); Eucalyptus smithii R. T. Baker, gully gum, gully peppermint,
blackbutt peppermint)
Eucalyptus macarthurii, Woolly butt, Camden woollybut, Paddy's river box (Eucalyptus macarthurii
H. Deane & Maiden)
Eucalyptus peppermint, Broad-leaved peppermint, blue peppermint, peppermint gum (Eucalyptus dives
Schauer in Walp.)
Fennel (Foeniculum vulgare Mill.)
Feverfew,, Nosebleed, midsummer daisy (Tanacetum parthenium (L.) Sch. Bip.,
Chrysanthemum parthenium (L.) Bernh.)
Fir Douglas (Pseudotsuga menziesii (Mirbel) Franco)
Fir cones silver, White fir, silver spruce, templin (Abies alba Mill.)
Fir needle Canadian, Balsam fir, fir balsam (Abies balsamea L.)
Fir needle Himalayan, East Himalayan fir, Himalayan silver fir (Abies spectabilis (D. Don) Spach,
Abies webbiana (Wall ex D. Don) Lindl., Pinus spectabilis D. Don, Pinus webbiana Wall ex D. Don)
Fir needle Japanese, Japanese pine needle, sachalin fir (Abies sachalinensis F. W. Schmidt)
Fir needle Siberian, Russian fir, Siberian silver fir, Siberian pine needle (Abies sibirica Ledeb.)
Fir needles silver, White fir, silver spruce, templin (Abies alba Mill.)
Frankincense, Olibanum (Boswellia frereana Birdwood (synonym: Boswellia hildebrandtii Engl.) (African
elemi, elemi frankincense); Boswellia papyrifera (Del.) Hochst (synonyms: Amyris papyrifera,
Boswellia chariensis, Boswellia occidentalis, Boswellia odorata, Ploesslea floribunda) Sudanese frankincense;
Boswellia sacra Flueck. (synonyms: Boswellia carteri Birdwood, Boswellia bhau-dajiana Birdwood,
Boswellia undulatocrenata) Saudi frankincense; Boswellia serrata Roxb. (synonyms: Boswellia glabra
Roxb., Boswellia thurifera Roxb., Chloroxylon dupada) Indian frankicnese; Boswellia neglecta S. Moore
(‘Borena’ type frankincense); Boswellia rivae Engl. (‘Ogaden’ type frankincense))
Galangal greater, Siamese ginger (Alpinia galanga L., Amomum galanga L., Languas vulgare Koenig.,
Languas galanga L.)
Galbanum (Ferula galbaniflua Boiss. & Buhse, Ferula gummosa Boiss., Ferula erubescens Boiss.)
Garlic (Allium sativum L.)
Geranium (Pelargonium × asperum)
Ginger (Zingiber officinale)
Gingergrass, Russa grass, sofia (Cymbopogon martinii, Andropogon martinii)
Ginger lily, White ginger lily, butterfly lily, (Hedychium coronarium)
Grapefruit (Citrus × paradisi Macfady)
Hemp, Cannabis (Cannabis sativa L., Cannabis indica L.)
Hibawood, Hiba, false arborvitae (Thujopsis dolobrata)
Hinoki leaf (Chamaecyparis obtusa (Siebold & Zucc.) Endl. var. obtusa, Cupressus obtusa)
Hinoki root (Chamaecyparis obtusa (Siebold & Zucc.) Endl. var. obtusa, Cupressus obtusa)
Hinoki wood (Chamaecyparis obtusa (Siebold & Zucc.) Endl. var. obtusa, Cupressus obtusa)
Ho leaf, Hon-sho, true camphor tree (Cinnamomum camphora L.)
Honeysuckle (Lonicera periclymenum L and other Lonicera species such as L. caprifolium L. and L. etrusca Santi)
Hop, Common hop (Humulus lupulus L.)
Hyssop (Hyssopus officinalis L. var. decumbens Briq.)
Immortelle, Everlasting, helichrysum, curry plant (Helichrysum italicum (Roth) G. Don (synonym:
Helichrysum angustifolium (Lam.) DC; Helichrysum stoechas L. subsp. stoechas)
Inula, Sweet inula, camphor inula (Dittrichia graveolens (L.) Greuter, Inula graveolens (L.) Desf.)
Jasmine, Royal jasmine, Spanish jasmine (Jasminum grandiflorum L., Jasminum officinale L. var.
grandiflorum L., Jasminum officinale L. f. grandiflorum L.)
Jasmine sambac, Arabian jasmine (Jasminum sambac L.)
Juniperberry, Juniper (Juniperus communis L.)
Juniper Phoenician, Phoenician savin (Juniperus phoenicea L.)
Kanuka, White tea tree, burgan (Kunzea ericoides (A. Rich.) Joy Thomp., Kunzea peduncularis F.
Muell., Leptospermum ericoides A. Rich)
Labdanum, Cistus ladanifer L. (Cistus ladaniferus Curtis)
Lantana (Lantana camara L.)
Larch needle, American larch, larch tamarack, hackmatack (Larix laricina Du Roi)
Laurel berry (Laurus nobilis L., Laurus novocanariensis (previously Laurus azorica))
Laurel leaf, Bay leaf, bay laurel, sweet bay (Laurus nobilis L.)
Lavandin (Lavandula × intermedia Emeric ex Loisel., Lavandula hybrida Reverchon, Lavandula hortensis Hy)
Lavender, Common lavender, English lavender (Lavandula angustifolia Mill., Lavandula officinalis Chaix
in Villars, Lavandula vera DC)
Lavender Spanish, French lavender, maritime lavender (Lavandula stoechas L. ssp. stoechas)
Lavender spike, Spike (Lavandula latifolia Medic., Lavandula spica DC)
Leek (Allium porrum L.)
Lemon balm Australian, Lemon-scented ironbark (Eucalyptus staigeriana F. v. Muell. ex F. M. Bailey)
Lemon (Citrus × limon L., Citrus limonum Risso)
Lemongrass (Cymbopogon flexuosus Nees ex Steud. (synonym: Andropogon flexuosus Nees ex Steud.)
(East Indian); Cymbopogon citratus DC (synonym: Andropogon citratus DC) (West Indian))
Lemon-scented gum, Eucalyptus citriodora, lemon-scented iron gum, spotted gum, lemon eucalyptus
(Corymbia citriodora Hook., Eucalyptus citriodora Hook., Eucalyptus maculata Hook. var. citriodora
Hook., Eucalyptus melissiodora Lindley)
Lime, West Indian lime, Key lime, Lime Tahiti (Citrus × latifolia Tanaka, Citrus × aurantifolia Christm.)
Linaloe wood (Bursera glabrifolia Humb. (synonym: Bursera delpechiana Poisson) and other Bursera
species such as B. aloexylon Schiede, B. fagaroides Humb., B. penicillata Sesse & Moc. and B. simaruba L))
Longoza, Longozo (Hedychium coronarium)
Lovage leaf (Levisticum officinale W. S. Koch)
Lovage root (Levisticum officinale)
Lovage seed (Levisticum officinale)
Mace (Myristica fragrans Houtt., Myristica officinalis L. fil., Myristica moschata, Thunb.,
Myristica aromatica O. Schwartz, Myristica amboinensis Gand.)
Magnolia flower, White champaca, white jade orchid (Michelia alba DC, Michelia longifolia Blume,
Sampacca longifolia (Blume) Kuntze)
Magnolia leaf, White champaca, white jade orchid (Michelia alba DC, Michelia longifolia Blume,
Sampacca longifolia (Blume) Kuntze)
Mandarin, Common mandarin (Citrus reticulata Blanco, Citrus nobilis Andrews)
Mandarin leaf, Mandarin petitgrain, petitgrain mandarin (Citrus reticulata Blanco, Citrus nobilis Andrews)
Mango ginger (Curcuma amada Roxb.)
Manuka (Leptospermum scoparium J. R. Forster & G. Forster)
Maraba, False galangal, false ginger, small galangal, resurrection lily (Kaempferia galanga L.)
Marjoram Spanish, Mastic thyme, Spanish wild marjoram (Thymus mastichina L. ssp. mastichina)
Marjoram sweet (Origanum majorana L., Botanical synonyms: Majorana hortensis Moench, Origanum dubium Boiss.)
Massoia (Cryptocarya massoy (Oken) Kosterm., Cryptocaria massoia (Becc.) Kosterm., Massoia aromatica Becc.)
Mastic (Pistacia lentiscus L.)
May chang, Pheasant pepper tree (Litsea cubeba (Lour.) Pers., Litsea citrata Blume, Laurus cubeba Lour)
Melissa, Balm, lemon balm (Melissa officinalis L.)
Mimosa (Acacia dealbata Link, Acacia decurrens Willd. var. dealbata (Link) Muller,
Racosperma dealbatum (Link) Pedley)
Mint bergamot (Mentha aquatica L. var. citrata (Her.) Fresen, Mentha citrata Ehrh.)
Mint wild forest, Common horsemint common (Mentha longifolia L.)
Mugwort common, camphor/thujone CT, Mugwort, Indian wormwood (Artemisia vulgaris L.)
Mugwort Douglas, Douglas sagewort, California mugwort (Artemisia douglasiana Bess.)
Mugwort great (Artemisia arborescens L.)
Muhuhu (Brachylaena huillensis O. Hoffm., Brachylaena hutchinsii Hutch.)
Myrrh, Somalian myrrh (Commiphora myrrha (Nees) Engl., Commiphora molmol Engl.)
Myrtle (Myrtus communis L.)
Myrtle aniseed, Anise-scented myrtle (Backhousia anisata Vickery)
Myrtle honey, Marsh honey myrtle (Melaleuca teretifolia Endl.)
Myrtle lemon, Lemon-scented myrtle, lemon ironwood, sweet verbena tree (Backhousia citriodora F. Muell.)
Nagarmotha, Cypriol, cyperus (Cyperus mitis Seud., Cyperus scariosus R. Br.)
Narcissus, Poet's narcissus (Narcissus poeticus L.)
Nasturtium, Indian cress (Tropaeolum majus L.)
Neroli, Orange blossom (Citrus × aurantium L.)
Niaouli (Melaleuca quinquenervia Cav.)
Nut grass, Coco grass, motha (Cyperus rotundus L.)
Nutmeg (Myristica fragrans Houtt., Myristica officinalis L. fil., Myristica moschata Thunb.,
Myristica aromatica O. Schwartz, Myristica amboinensis Gand.)
Onion (Allium cepa L.)
Opopanax, Opoponax, scented myrrh, sweet myrrh, bisabol myrrh (Commiphora guidottii Chiov.)
Orange, Seville orange (Citrus sinensis L., Citrus aurantium L. var. sinensis, Citrus × aurantium L.,
tanical synonyms: Citrus aurantium L. subsp. amara L., Citrus aurantium L. subsp. aurantium)
Orange flower (Citrus sinensis L., Citrus aurantium L. var. sinensis, Citrus × aurantium L., tanical
synonyms: Citrus aurantium L. subsp. amara L., Citrus aurantium L. subsp. aurantium)
Orange leaf, Petitgrain, orange petitgrain, bitter orange leaf, sour orange leaf, Seville orange leaf
(Citrus × aurantium L.)
Oregano, Origanum (Origanum syriacus L., Origanum onites L. (synonym: Origanum smyrnaeum L.);
Origanum vulgare L. subsp. hirtum (Link) letswaart (synonyms: Origanum compactum, Origanum hirtum
Link); Thymbra capitata (L.) Cav. (synonyms: Thymus capitatus L., Coridothymus capitatus L.,
Satureja capitata L.); and occasionally other Origanum species)
Oregano Mexican (Lippia graveolens HBK, Lippia berlandieri Schauer, Lippia amentacea M. E. Jones,
Lippia tomentosa Sessé & Moc.)
Orris, Iris (Iris pallida Lam., Iris × germanica L.)
Osmanthus, Fragrant olive, sweet olive, tea olive (Osmanthus fragrans Lour., Olea fragrans Thunb.)
Palmarosa, Motia, rosha grass (Cymbopogon martinii Roxb. var. martinii, Andropogon martinii Roxb. var
martinii, Cymbopogon martinii Roxb. var. motia)
Palo santo (Bursera graveolens (Kunth) Triana et Planch)
Parsley leaf, Parsley herb (Petroselinum crispum Mill., Petroselinum sativum Hoffm., Petroselinum hortense auct.)
Parsley seed (Petroselinum crispum Mill., Petroselinum sativum Hoffm., Petroselinum hortense auct.)
Parsnip (Pastinaca sativa L.)
Patchouli (Pogostemon cablin (Blanco) Benth., Pogostemon patchouly Pellet)
Pepper (Piper nigrum L.)
Pepper pink, California pepper, Peruvian pepper, Peruvian mastic (Schinus molle L.)
Pepper Sichuan, Japanese pepper, fagara, prickly ash, hua jiao (Zanthoxylum piperitum DC)
Peppermint (Mentha × piperita L.)
Perilla, Beefsteak plant, shiso (Perilla frutescens (L.) Britt.)
Peru balsam (Myroxylon balsamum (L.) Harms var. pereirae (Royle) Harms, Myroxylon pereirae Royle;
Myroxylon peruiferum L. F. Myrospermum pereirae Royle; Toluifera pereirae Royle)
Peta (Helichrysum splendidum Less.)
Phoebe (Phoebe porosa Mez., Oreodaphne porosa Nees ex Mart)
Pimento leaf, Pimenta leaf (Pimenta dioica L., Pimenta officinalis Lindl.)
Pine black (Pinus nigra J. F. X Arnold)
Pine dwarf, Mountain pine, dwarf mountain pine, mugo pine, pumilio pine (Pinus mugo Turra (synonym:
Pinus Montana Mill.); Pinus mugo Turra var. pumilio Haenke (synonym: Pinus pumilio Haenke);
Pinus mugo Turra var. mugo Zenari (synonym: Pinus mugus Scop.))
Pine grey, Jack pine (Pinus divaricata Alton, Pinus banksiana Lamb.)
Pine ponderosa, Western yellow pine (Pinus ponderosa Douglas ex P. Lawson & C. Lawson)
Pine, Scotch pine (Pinus sylvestris L.)
Pine white (Pinus strobus L.)
Piri-piri, Jointed flat sedge, priprioca, adrue (Cyperus articulatus L.)
Plai (Zingiber montanum (J. König) Theilade, Amomum montanum J. König, Zingiber cassumunar
Roxb.; Zingiber purpureum Roscoe)
Plectranthus (Plectranthus fruticosus L'Hérit.)
Rambiazana, Rambiazinza vavy (Helichrysum gymnocephalum Humbert)
Ravensara bark, Havozo (Ravensara aromatica Sonnerat, Ravensara anisata Danguy et Choux)
Ravensara leaf (Ravensara aromatica Sonnerat, Ravensara anisata Danguy et Choux)
Rhododendron, Anthopogon, sunpati (Rhododendron anthopogon D. Don.)
Rosalina, Lavender tea tree, Swamp paperbark tree (Melaleuca ericifolia Smith)
Rose Damask, Rose otto (Rosa × damascena Mill., Rosa damascena Mill. var. trigintipetala (Dieck)
Koehne; Rosa gallica f. trigintipetala Dieck)
Rose Japanese, Ramanas rose, rugosa rose (Rosa rugosa Thunb.)
Rose Provence, Cabbage rose, rose de Mai, French rose, hundred-leaved rose (Rosa × centifolia L.,
Rosa gallica L. var. centifolia (Regel.))
Rosemary (Rosmarinus officinalis L.)
Rosewood, Bois de rose, Pau-rosa (Aniba rosaeodora Ducke, Aniba amazonica Ducke, Aniba parviflora
Meissner Mez.)
Saffron (Crocus sativus L.)
Sage blue mountain, Stenophylla (Salvia stenophylla Burch, ex Benth.)
Sage Greek, Turkish sage (Salvia fruticosa Mill., Salvia triloba L.)
Sage Spanish, Lavender sage (Salvia lavandulifolia Vahl., Salvia hispanorum Lag.)
Sage white (Salvia apiana Jeps.)
Sage wild mountain, Mountain salvia (Hemizyqia petiolata Ashby)
St. John's wort (Hypericum perforatum L.)
Sandalwood East African (Osyris lanceolata Hochst. & Steud.; Osyris tenuifolia Engl.)
Sandalwood New Caledonian, Pacific island sandalwood (Santalum austrocaledonicum Vieill.)
Sandalwood Western Australian (Santalum spicatum A. DC., Santalum cygnorum Miq; Fusanus spicatus
R. Br.; Eucarya spicata Sprag, et Summ.)
Sanna, Spiked ginger lily, kapur kachri (Hedychium spicatum Sm.)
Saro, Mandravasarotra (Cinnamosma fragrans Baill.)
Satsuma, Mikan (Citrus unshiu Marc., Citrus reticulata Blanco var. satsuma)
Savory (Satureia hortensis L. (summer savory); Satureia montana L. (winter savory))
Southernwood (Artemisia abrotanum L.)
Spearmint (Mentha cardiaca G. (Scotch spearmint); Mentha spicata L. var. crispa (Bentham) Danert
(native spearmint), Mentha crispa L., Mentha viridis L. (native spearmint))
Spruce black, Canadian black ‘pine’ (Picea mariana (Mill.) Britton, Picea nigra Link)
Spruce hemlock, Eastern hemlock, hemlock (Tsuga Canadensis (L.) Carrière, Pinus canadensis L.,
Picea canadensis L.)
Spruce Norway, Common spruce, European spruce (Picea abies L., Picea excelsa Link)
Spruce red (Picea rubens Sarg.)
Spruce white, Canadian spruce (Picea glauca (Moench) Voss, Picea alba Link, Picea canadensis Mill.)
Styrax, Storax, sweetgum (Liquidambar orientalis Mill. (Asian styrax), Liquidambar styraciflua L. var
macrophylla (American styrax))
Sweet vernalgrass, Flouve (Anthoxanthum odoratum L.)
Taget, Marigold, tagetes (Tagetes minuta L. (synonym: Tagetes glandulifera Schrank), Tagetes patula L.)
Tana, Issa (Rhus taratana (Baker) H. Perrier)
Tangelo (Citrus reticulata Blanco × Citrus × paradisi Macfady)
Tangerine (Citrus reticulata Blanco, Citrus nobilis Andrews, Citrus tangerine Hort. ex Tanaka)
Tansy (Tanacetum vulgare L., Chrysanthemum tanacetum Karsch, Chrysanthemum vulgare L.)
Tansy blue, Moroccan Tansy, Moroccan blue chamomile (Tanacetum annuum L.)
Tea leaf, Black tea (Camellia sinensis L., Thea sinensis L.)
Tea tree (Melaleuca alternifolia Cheel (syn. Melaleuca linariifolia var. alternifolia Maid. & Bet.))
Tea tree black, River tea tree oil (Melaleuca bracteata F. von Müller)
Tea tree lemon-scented, Lemon tea tree (Leptospermum petersonii F. M. Bailey (syn:
Leptospermum citratum Chalk, Cheel & Pent.); Leptospermum liversidgei R. T. Baker & H. G. Smith)
Tejpat, Indian cassia (Cinnamomum tamala Buch.-Ham.)
Thyme, Moroccan thyme, thyme spike (Thymus vulgaris L., Thymus satureioides Coss. & Bal.,
Thymus zygis L., Thymbra spicata L.)
Thyme lemon (Thymus × citriodorus (Pers.) Schreb., Thymus lanuginosus Mill. var. citriodorum Pers.,
Thymus serpyllum var. citriodorus (Hort.), Thymus serpyllum L. var. vulgaris Benth.)
Tolu balsam, Thomas balsam, opobalsam (Myroxylon balsamum (L.) Harms var. balsamum)
Tonka (Dipteryx odorata (Aubl.) Willd., Coumarouna odorata Aubl.)
Turmeric leaf (Curcuma longa L.)
Turmeric rhizome, Indian saffron (Curcuma longa L., Curcuma domestica Vai., Curcuma aromatica Salisb.)
Valerian European type (Valeriana officinalis L., Valeriana fauriei Briq.)
Valerian Indian, Sugandhawal (Valeriana jatamansi Jones, Nardostachys jatamansi (D. Don) DC;
Valeriana wallichii DC, Fedia grandiflora Wall.; Patrinia jatamansi (Jones) D. Don)
Vanilla (Vanilla planifolia Andr. (synonym: Vanilla fragrans Salisb.) (Bourbon vanilla); Vanilla tahitensis J.
W. Moore (Tahitian vanilla))
Verbena honey, Wild verbena, greendog, zinziba (Lippia javanica Spreng.)
Verbena lemon, Verbena (Aloysia triphylla L'Hérit., Aloysia citriodora Ortega ex Pers., Lippia citriodora
Ortega ex Pers., Lippia triphylla L'Hérit., Lippia alba (Mill.) N. E. Brown)
Vetiver, Khus, khus-khus (Vetiveria zizanoides (L.) Nash, Andropogon muricatus (Retz.),
Andropogon zizanoides (L.) Urban, Chrysopogon zizanoides (L.) Roberty, Phalaris zizanoides L.)
Wintergreen (Gaultheria fragrantissima Wall.; Gaultheria procumbens L.)
Wormwood, Absinthe, artemisia (Artemisia absinthium L.)
Wormwood annual, Sweet wormwood, sweet annie (Artemisia annua L.)
Yarrow, Milfoil (Achillea millefolium L.)
Ylang-ylang (Cananga odorata J. D. Hook. & T. Thompson f. odorata, Cananga odorata J. D. Hook. & T.
Thompson f. genuina)
Yuzu (Citrus junos Sieb. ex Tanaka.)
Zedoary, White turmeric, hidden ginger (Curcuma zedoaria Roscoe)

Herbs that may be used in accordance with the present invention include those mentioned herein, as well as other herbs that may be used as a therapy or treatment for an illness or disease when modified to be in microparticle form_Non-limiting examples of herbs that may be used in accordance with embodiments of the present invention include the following:

Achyrantis (Radix Achryranthis Bidentatae)
Aconite (Aconitum Kusnezoffii, Aconitum spp.)
Acorus (acorus calmus, Rhizoma Acori Tatarinowii )
Agaricus mushroom
Agarikon mushroom
Agimony (Herba Agrimoniae)
Akebia (Caulis Akebiae)
Alisma (Rhizoma Alismatis)
Amomum (Fructus Amomi)
Andrographis
Angelica dahuricae radix
Angelica sinensis radix
Artemisia annua L.
Artemisia argyii folium
Ashwagandha
Asparagus root (Radix Asparagi)
Astragalus root
Bamboo sap (Caulis Bambusa in Taenia)
Basil leaf
Belamcanda (Belamcanda Chinensis Rhizome)
Bitter melon
Bacopa monniera
Black walnut hull
Bauhinia tormentosa
Burdock root
Cascara sagada
Cats claw
Centella asiatica
Chaga mushroom
Chrysanthemum flower (Flos Chrysanthemi)
Lovage root (Chuanxiong Rhizoma)
Cimcifuga (Rhizoma Cimcifugae)
Cinnamon bark
Cinnamon twig (Cinnamomi Ramulus)
Cistus incanus
Citrus seed (Semen Citri Reticulatae)
Clerodendrum Trichotomum
Club moss
Clove bud
Cnidium
Coptis Chinensis
Cordyceps mushroom,
Cornus (Cornus Officalis Fructus)
Corydalis (Corydalis Rhizome)
Curcuma root (Curcuma Phaeocaulis Radix)
Cynomorus (Herba Cynomorii)
Cyperus (Rhizoma Cyperi)
Cortex Lycii
Crataegus (Fructus Crataegi)
Curcurmae Rhizoma
Cyanchi (Radix et Rhizoma Cyanchi Paniculati)
Dandelion root
Deer antler (Cornu Cervi Pantotrichum)
Dichroae Radix
Dioscorea root (Rhizoma Dioscoreae)
Dipsacus (Dipsaci Radix)
Dragon bone (Os Draconis)
Dryopteridis (Cyrtomii Rhizoma)
Echinacea purpurea
Eclipta (Ecliptae Prostratae Herba)
Eleuthero root
Elsholtzia (Elsholtziae Splendentis Herba)
Epimedium leaf
Eribotrya leaf (Eriobotryae Japanicae Folium)
Eucommia (Cortex Eucommiae)
Evodia (Evodia Rutaecarpa Frctus)
Fenugreek seed
Forsythia fruit
Frankincense (Gummi Olibanum)
Fritillaria (Fritillaria Thungbergii Bulb)
Fructus Jujubae
Fructus Psorleae
Fructus Toosendan
Fructus Tsaoko
Chinese nut gall (Galla Chinensis)
Gardenia (Gardenia Jasminoides Fructus)
Gastrodia (Rhizoma Gastrodiae)
Gentiana Macrophyla root (Gentiana Macrophyllae Radix)
Gentiana scabra root (Gentiana Scabra Radix)
Ginger root (Zingiberis Recens Rhizoma)
Ginseng root (Ginseng Radix et Rhizoma)
Gynostemma (Gynostemma Fructus)
Gypsum (Gypsum Fibrosum)
He Shou Wu, Fo-Ti (Polygonum Multiflorum Radix)
Herba cum radice patriniae
Herba Menthae
Herba Scutellariae Barbarate
Honeysuckle flower (Lonicera Japonica Flos)
Honeysuckle vine (Lonicera Japonica Caulis)
Houttuynia (Houttuyniae Herba)
Indigo (Indiogofera Tinctoria L.)
Isatis leaf (Baphicacanthus Folium)
Isatis root
Jave brucea fruit
Kudzu root
Licorice root (Radix Glycyrrhiza glabra)
Ligustrum (Fructus Ligustri Lucidi)
Lily bulb
Lindera (Radix Linderae)
Lions mane mushroom
Lithospermum (Arnebia Euchroma Radix)
Longan fruit (Arillus Longan)
Lotus rhizome (Nodus Nelumbinis Rhizomatis)
Lycii berries
Lycopus (Lycopus Lucidus Herba)
Magnetitum
Magnolia Bark (Magnolia Officianlis Bark)
Magnolia flower (Magnolia Officianlis Flos)
Maitake mushroom
Milk thistle
Mimosa pudica
Mistletoe
Mucuna puriens
Mulberry fruit (Fructus Mori)
Mulberry leaf (Morus Alba Folium)
Mulberry twig (Morus Alba Ramulus)
Myrrh (Commiphora Myrrha)
Neem leaf
Nettle leaf
Prunella (Spica Prunellae)
Pseudoginseng, notoginseng (Notoginseng Radix et Rhizoma)
Nutmeg (Semen Myristicae)
Olive leaf
Oldenalndia (Oldenalndia diffusa)
Ophiopogon (Radix Ophiopogonis)
Oregon grape root
Osha root (Ligusticum Por. Osha)
Oyster shell (Ostrea Gigas)
Patchouli (Pogostemon Cablin Herba)
Peach kernel (Semen Persicae)
White peony root (Peoniae lactiflora radix)
Perilla (Perilla Folium)
Phellodendron (Phellodendron Radix)
Earthworm (Pheretima)
Pineilia prepared
Plantaginis Herba
Poke root
Polygala
Polygonum (Polygoni Multiflori Caulis)
Polygonatum Rhizome (Polygonati Rhizoma)
Japanese Knotweed (Polygonum cuspidatae)
Lysimachia (Herba Lysimachiae)
Litchi seed (Semen Litchi)
Platycodon root (Radix Platycodonis)
Poria fungus
Poria Paradicis
Privet fruit
Quassia bark
Radix Angelica Pubscentis
Radix Atractylodis Macrocephalae, Radix Atractylodis
Radix Cyanthulae
Radix Glehniae
Radix Peonae, Radix Peonae Rubrae
Radix Puerariae Lobatae
Radix Pulsatilla
Rehmannia raw, cooked (Radix Rehmanniae)
Radix Saposhnikoviae
Radix Stephaniae Tetrandrae
Radix Vladimiriae
Ramulus Uncariae cum Uncis
Red sage, Chinese sage, tan shen, or danshen (Radix Salvia miltiorrhiza)
Reishi mushroom
Reishi mushroom spores
Rhizoma Alpiniae Officinarum
Rhizoma Drynariae
Rhodiola rosea
Safflower (Flos Carthami)
Sanguisorbia Officinalis Radix
Schisandra berry (Schisandra Chinensis Fructus)
Schizonepeta (Herba Schizonepetae)
Scrophularia
Scutellaria root (Scutellariae Radix)
Semen Platycladi
Shiitake mushroom
Sida cordifolia
Smilax (Smilacis Glabrae Rhizome)
Sophora flower(Sophora flavescens Flos)
Sophora root (Sophora flavescens Radix)
Sour plum
Spargani (Rhizoma Sparganii)
Spatholobus root, vine (Spatholobi Caulis)
St. John's Wort
Tangerine peel
Taxilia (Herba Taxilli)
Teasel root american
Terminalia chebula
Thunder god vine
Tree Peony (Moutan Radicis Cortex)
Tribulus (Tribulus terrestris Fructus)
Tricosanthis (Tricosanthes Kirlowii Radix)
Turkey tail mushroom
Turkey rhubarb (Rheum palmatum, Radix Rhizoma Rhei)
Turmeric root (Curcuma longa)
Verbena (Herba Verbenae)
Viola (Viola Yedoensis Herba)
Vitex (Vitex negundo)

Supplements that may be used in accordance with the present invention include those mentioned herein, as well as other supplements that may be used as a therapy or treatment for an illness or disease when modified to be in microparticle form. Non-limiting examples of supplements that may be used in accordance with embodiments of the present invention include the following:

Acacia fiber
Benfotiamine
Colloidal gold
Colloidal silver
Curcumin
Erythritol
Glutamine
Glutathione
Glycerine
Grapefruit seed extract
Hemp extract, CBD
Huperzine
Ionic silver
Lysozyme
Magnesium
Monkfruit extract
NAC
Phosphatidylcholine
Phosphatidylserine
Potassium sorbate
Propolis
Resveratrol
Seawater, Quintons
Sodium Butyrate
Stevia leaf extract
Sunflower lecithin
Vitamin C
Xylitol

The present compositions may include one or more active substances (such as essential oils, herbs and supplements), which are included to achieve the desired therapeutic effect, and may optionally also include one or more excipients, such as a carrier, coating, binder, etc, as may be determined by those skilled in the art, so long as the therapeutic effect is not compromised by virtue of the excipient(s).

The term “excipient” is used herein to include pharmaceutically acceptable inert substances added to a drug formulation or composition to give e.g., a desired consistency or form, or used as a carrier. Non-limiting examples of excipients that may be included in the present compositions and/or formulations herein may include, but are not limited to binders, fillers, diluents, lubricants, anti-infectious agents, antimicrobial agents and solubility modifiers, and other excipients known to those skilled in the art, depending e.g., on the composition being formed, intended method of administration and/or method of formation, the active ingredient(s) being used, etc.

Non-limiting examples of the present invention include novel methods of using FDA GRAS microparticle compositions to treat infections, such as tick infections, in humans and animals.

According to example embodiments, the present invention provides microparticle compositions that include one or more ingredients selected from essential oils, herbs, and supplements, that have been modified or synthesized to be in microparticle form. The one or more essential oils, herbs, and supplements in microparticle form are formulated to be pharmaceutically acceptable for administration in an effective amount to a subject, for treatment of an infection or disease in the subject. In non-limiting example embodiments, the infection or disease is a Lyme tick borne illness related infection or disease, or a coronavirus.

Non-limiting examples of infections that may be treated using the present compositions and methods may include bacterial, viral and/or fungal infections. By way of non-limiting example, the invention may include one or more Lyme tick infections. Infections that may be treated by the present compositions and methods may include for example Borrelia, Bartonella, Babesia, Ehrlichia, Anaplasmosis, Rickettsia, Mycoplasma, viruses, parasites, fungi, bacteria, and any other tick infection. It is also contemplated that the infection may be a coronavirus infection, such as Covid-19.

By way of non-limiting example, the microparticle composition of the present invention may include one or more of the following ingredients: Carrot seed essential oil (EO), celery seed EO, cinnamon bark, clove bud EO, German chamomile EO, copaiba EO, geranium EO, helichrysum EO, lemongrass EO, mountain savory EO, oregano EO, spearmint EO, organic sunflower lecithin, Roman chamomile EO, cardamom seed EO, cinnamon EO, lavender EO, lemon balm, thyme EO, organic sunflower lecithin, Bergamot EO, hyssop EO, marjoram EO, nutmeg EO, petitgrain EO, and thyme EO. It may further include purified water. Compositions including these components may be used for example for the treatment of Lyme tick borne infections, illness or disease.

By way of non-limiting example, the microparticle composition of the present invention may include one or more of the following ingredients: Bay Laurel EO, Eucalyptus globulus EO, organic sunflower lecithin, Herba houttuyniae, Ganoderma, Folium isatidis, Radix scutellariae, Galla chinensis, Radix astragali, Radix et Rhizoma glycyrrhizae, Folium mori, Flos chrysanthemi, Flos lonicerae japonica, Fructus forsythia, Herba moslae, Rhizoma zingiberis, and Folium eriobotryae. The composition may further include purified water, such as reverse osmosis water. Compositions including these components may be used for example for the treatment of coronovirus infections, illness or disease.

According to example embodiments, the one or more essential oils, herbs, and supplements, are modified or synthesized into one or more microparticle forms selected from liposomes, nanoparticles, nanostructured lipid carriers, and nano-emulsion compositions.

Methods of treatment in accordance with non-limiting example embodiments of the present invention may include treating a subject/patient having an infection with one or more of the present microparticle therapy compositions, by administering an effective amount of the composition(s) to a subject/patient in need of such treatment. According to non-limiting example embodiments, it is contemplated that methods of treatment may include treating a subject or patient who may not yet have an infection, as a preventative treatment. Non-limiting example methods may have one or more of the following effects to be considered treatment, lessening or eliminating the infection or disease or symptoms thereof, reducing the duration of the infection or disease, or symptoms thereof, and/or lessening or reducing the possibility of catching the infection or disease.

Non-limiting example embodiments of the present invention include methods of treating a subject having an infection or disease, such as a Lyme tick borne illness related infection or disease or a coronavirus infection, which include administering to the subject a microparticle composition provided herein. The microparticle compositions herein may include one or more ingredients selected from essential oils, herbs, and supplements, that have been modified or synthesized to be in microparticle form, in an effective amount to treat the Lyme tick borne illness related infection or disease or a coronavirus infection.

Appropriate methods of administration may include for example, oral administration in a liquid, tablet, capsule, sublingual or other oral form, as would be known to those skilled in the art. For example, essential oils can be taken internally in liposomes, in a carrier oil, or in a suppository: sublingual, oral, nasal, otic, rectal, or vaginal. The target may be neurological, sinus, oral, respiratory, pelvic, systemic symptoms. Essential oils may also be applied externally, such as topically, by laser remedy, or diffuse to target the respiratory, skin, joint, muscular and systemic symptoms. Other non-limiting methods of administration, such as IV and injection methods are also contemplated as being suitable. In example embodiments, the microparticle compositions of the present invention may be administered to a subject by an administration method selected from orally, nasally, rectally, vaginally, topically, intravenously or by injection.

According to example methods, the one or more essential oils (EOs), herbs, and supplements; are modified into one or more microparticle forms selected from the group consisting of liposomes, nanoparticles, nanostructured lipid carriers, and nano-emulsion compositions.

According to non-limiting example embodiments, the infection or disease may be a Lyme tick-borne infection or disease, such as drug-resistant forms, drug-persisters, biofilms, cystic forms of tick infections including Borrelia, Bartonella, Babesia, Ehrlichia, Anaplasmosis, Rickettsia, Mycoplasma, viruses, parasites, fungi, bacteria, and any other tick infection. It is also contemplated that the infection may be a coronavirus infection, such as Covid-19.

According to example embodiments, the formulation and treatment may be custom engineered to the patient depending on the patients underlying issues.

The present therapies or compositions may be administered by any method deemed suitable by a physician or other individual skilled in the art. As indicated above, by way of non-limiting example, the composition/therapy may be administered orally, nasally, rectally, vaginally, topically intravenously or by injection.

As used herein, the term “recipient”, “subject” or “patient” is intended to include any animal, such as a mammal (including, but not limited to humans) to whom the present compositions may be administered. A subject or patient may or may not be under current medical care, and may or may not have had one or more prior treatments. Although, as would be apparent to those skilled in the art, the formulations and dosages may be different for non-humans than for humans, taking into consideration certain requirements for safety for administration.

Appropriate dosages of the therapies or compositions provided herein may be determined by those skilled in the art, depending on various factors, such as the severity of the infection or other ailment being treated, the weight of the subject, the type of infection or other ailment being treated, etc. Dosage amounts, frequency, and total number of doses, may be adjusted to achieve desired affects, depending on for example, the subject's/tolerance and reaction to previous doses (if any). A unit dosage may comprise a therapeutically effective amount of one or more ingredients. A unit dosage will depend upon many factors, which may include for example, age, size, and condition of the individual being treated and the number of times the unit will be taken.

According to example embodiments, essential oils may be used for targeting persistent lyme disease and co-infections. Anti-microbial essential oils may be used to fight or inhibit for example Borrelia persisters and chronic symptoms in reservoirs: brain, joints, inside cells, and under biofilms. The essential oils may also be used for targeting coinfections, and/or for targeting relapsing symptoms.

According to non-limiting example embodiments, compositions of microparticle essential oils may be effective to treat patients having Bartonella. In particular, microparticles of the following essential oils may be used to treat Bartonella.

Carrot seed EO about 3-5% or about 4%, celery seed EO about 7-9% or about 8%, cinnamon bark about 0.1-2% or about 1%, clove bud EO about 0.1%-2%, or about 1%, German chamomile EO about 7-9% or about 8%, copaiba EO about 7-9% or about 8%, geranium EO about 3-5% or about 4%, helichrysum EO about 3-5% or about 4%, lemongrass EO about 12-14% or about 13%, mountain savory EO about 3-5% or about 4%, oregano EO about 4-6% or about 5% spearmint EO 3-5% or about 4%, organic sunflower lecithin about 34-38% or about 36%. It should be apparent that some of these ingredients may be removed and/or others added. The microparticles may be prepared for example by combining the oils with sunflower lecithin and reverse osmosis water in a 20K+ RPM high speed blender. The resulting emulsion is poured into a glass container. The glass container is placed in a 20-60 Khz ultrasound water bath for 30 minutes. The resulting solution is approximately 80% essential oil liposomes and 20% essential oil emulsion.

According to other non-limiting example embodiments, compositions of microparticle essential oils may be effective to treat patients having Borrelia and/or Babesia. In particular, microparticles of the following essential oils may be used to treat Borrelia and Babesia infections: Roman chamomile EO about 7-9%, or about 8%, cardamom seed EO about 1-3% or about 2%, cinnamon EO about 0.1-2% or about 1%, helichrysum EO about 7-9% or about 8%, lavender EO about 7-9% or about 8%, lemon balm about 3-5% or about 4%, thyme EO about 11-13% or about 12%, organic sunflower lecithin about 35-39% or about 37%. As with other embodiments, it should be apparent that some of these ingredients may be removed and/or others added, such as purified water (or reverse osmosis water). The microparticles may be prepared for example by sonication.

According to other non-limiting example embodiments, compositions of microparticle essential oils may be effective to treat patients having Borrelia. In particular, microparticles of the following essential oils may be used to treat Borrelia infections: Bergamot EO about 1.5-3.5% or about 2.5%, carrot seed EO about 1.5%-3.5% or about 2.5%, Roman chamomile EO about 4-6% or about 5%, hyssop EO about 1.5-3.5% or about 2.5%, lemongrass EO about 4-6% or about 5%, marjoram EO about 9-11% or about 10%, nutmeg EO about 4-6% or about 5%, petitgrain EO about 1.5-3.5% or about 2.5%, thyme EO about 9-11% or about 10%, organic sunflower lecithin about 60%-70%, or about 65%. As with other embodiments, it should be apparent that some of these ingredients may be removed and/or others added, such as purified water (or reverse osmosis water). The microparticles may be prepared for example by sonication.

According to other non-limiting example embodiments, compositions of microparticle essential oils may be effective to treat patients having a coronavirus, such as Covid-19. In particular, according to example embodiments, microparticles of the following essential oils may be used to Covid-19 infections: Bay Laurel EO about 18-22%, or about 20%, Eucalyptus globulus EO about 13-17%, or about 15%, and organic sunflower lecithin about 60-70%, or about 65%. As with other embodiments, it should be apparent that some of these ingredients may be removed and/or others, such as purified water (or reverse osmosis water), added. The microparticles may be prepared for example by sonication.

According to other example embodiments, microparticles of the following essential oils may be used to Covid-19 infections: Herba houttuyniae about 7-9%, or about 8%, Ganoderma about 7-9% or about 8%, Folium isatidis about 3-5% or about 4%, Radix scutellariae about 3-5% or about 4%, Galla chinensis 3-5% or about 4%, Radix astragali about 3-5% or about 4%, and Radix et Rhizoma glycyrrhizae about 2-4% or about 3%

Other herbs with anti-Covid-19 properties which may be included in the above or other formulations used to treat patients with a Covid-19 infection include Folium mori, Flos chrysanthemi, Flos lonicerae japonica, Fructus forsythia, and/or Herba moslae, Rhizoma zingiberis, and Folium eriobotryae.

Organic sunflower lecithin in an amount of about 60-70%, or about 65% may also be used for the treatment of Covid-19, with purified water such as reverse osmosis water.

The present inventor discovered that microparticle essential oils may result in reducing Jarisch-Herxheimer reactions. Essential oils in lab studies were also shown to reduce Lyme disease persisters and their biofilms. Essential oils may be combined with antibiotics to enhance treatment outcomes and reduce persisters.

Clove bud essential oil may be used e.g., to inhibit Borrelia persisters, Fusarium spp, Aspergillus spp., Aspergillus aflatoxin B 1, Candida, E. coli, Streptococcus spp., Staphylodoccus spp, MRSA, Giardia, or helminths.

Certain essential oils may be effective to treat Borrelia persisters. These essential oils include oregano, cinnamon bark, and clove bud, which completely eradicated all viable persister cells without any regrowth in subculture in fresh medium. Other oils, such as citronell and wintergreen did not have this effect. Garlic, allspice, myrrh, hedycheim, and Litsea cubeba completely eradicated all B. burgdorferi stationary phase cells at 0.1%. Other essential oils failed to do so, as was shown by visible spirochetal growth.anti-malaria essential oils for Babesia include caraway (875 mg/day), nutmeg (28 mg/day) and myrtle (65 mg/day).

Anti-Bartonella essential oils include clove bud. Anti-Mycoplasma essential oils include tea tree and bergamot FCF. Anti-Brucella essential oils include oregano oil and cinnamon bark (200 mg/day).

The present application further relates to methods of making the present compositions. “Microparticle” essential oil, herbal, and supplement therapies may be advantageous in achieving reduced toxicity, the ability to penetrate more deeply inside cells, biofilms, and tissues, and greater antimicrobial efficacy compared to non-microparticle remedies. Non-limiting methods of making the present compositions may include combining the one or more essential oils, herbs, and supplements; and modifying the essential oils, herbs, and supplements into a microparticle form.

As with other embodiments herein, in these embodiments, microparticle compositions, therapies, or therapy compositions of the present invention, may include for example, liposome, nanoparticle, nanostructured lipid carriers (NLCs), or nano-emulsion composition. These microparticles can be produced via several methods, which may include, but are not limited to the following methods that are presently known or future methods to be developed.
1. Sonication uses high frequency ultrasound waves to break up essential oils, herbs, and/or supplements into smaller microparticles. These microparticles are in a solution of water and a lipid or other biocompatible liposome coating. The coating forms around the microparticles in the solution.
2. Methanol injection uses water that is poured into a concentrated lipid-ethanol-essential oils, herbs, and/or supplements solution, and then ethanol is removed in an evaporator. Dilution with water causes spontaneous formation of small and homogenous unilamellar vesicles from micellar aggregate. The size of liposomes can be controlled by the ratio of ethanol to water.
3. Freeze-thaw method uses a repeated freeze-thaw cycle of freezing in liquid nitrogen at −196 degrees C. followed by thawing at temperatures as high as 122 degrees F. produces microparticle liposomes. In lab experiments, the freeze-thaw method has produced optimal microparticles using as few as five cycles.
4. French Pressure Cell method uses pressures as high as 20,000 PSI where aqueous suspensions of lipid and essential oils, herbs, and supplements are placed within the chamber of a French pressure cell at room temperature and rapidly extruded at 20,000 psi through a small orifice.

It is contemplated that other methods of forming microparticles may be developed and would be included within the scope of the present invention.

According to example embodiments, combinations of desired essential oils, herbs and other ingredients are combined, and thereafter formed into microparticles (such as liposome, nanoparticle, nanostructured lipid carriers (NLCs), or nano-emulsion composition), as described herein, or as would be understood by those skilled in the art based on the present disclosure.

The exact composition of a particular microparticle composition, formulated for use in the present invention, may vary, depending e.g., the components to be used, the desired infection to be treated, the intended recipient, the method of administration (topical, oral, etc), and other factors indicated herein and/or known to those skilled in the art.

Sublingual Essential Oils (EOs) may be used for Relapsing symptoms. In non-limiting example embodiments, essential oils may be mixed with a carrier oil 1:10, and 10 drops are placed under the tongue. Another method of delivery is the patient inhales through the mouth, exhales slowly through the nose=EO “vaping”. Essential oils Target neurological, nasal, oral, throat, respiratory, and systemic symptoms. These delivery methods may provide rapid relief in minutes of patient's most urgent symptoms.

Lavender EO may be used for neurological symptoms of pain, headaches, swelling, inflammation, anxiety, racing thoughts, inability to concentrate, stress response—Lavender essential oil in a carrier oil 1:10, sublingual maximum adult dose 160 mg/day—Paired with breathing technique to engage parasympathetic.

Spearmint EO may be used for systemic burning, heat symptoms, muscle and joint pain, inflammation, rashes, headaches, nausea, vomiting, respiratory issues—Spearmint essential oil in a carrier oil 1:10, sublingual maximum adult dose 115 mg/day—Paired with breathing technique to engage parasympathetic.

Peppermint EO may be used for lack of focus, drowsiness, memory problems, inability to concentrate, heat symptoms, rashes—Peppermint essential oil in a carrier oil 1:10, sublingual maximum adult dose 152 mg/day—Paired with breathing technique to engage parasympathetic.

Lemongrass EO may be used for symptoms of anxiety, insomnia, seizures, digestion issues, mold toxicity, menstruation problems, bladder issues, headaches, cramps, rheumatism—Lemongrass essential oil in a carrier oil 1:10, sublingual maximum adult dose 46 mg/day—Paired with breathing technique to engage parasympathetic.

Eucalyptus globulus EO may be used for symptoms of inflammation, mold toxicity, respiratory issues, lack of focus, pain—Eucalyptus globulus essential oil in a carrier oil 1:10, sublingual maximum adult dose 600 mg/day—Paired with breathing technique to engage parasympathetic.

Each of these EOs may be contraindicated for certain patients having certain conditions. They may also cause side effects. A practitioner skilled in the art may be able to avoid or minimize side effects and/or administration of contraindicated EOs to patients, based on an examination of the patient and their history, and based on the present disclosure and information known in the art.

Within 24 hours after antibiotic treatment of the spirochetal infections syphilis, Lyme disease, leptospirosis, and relapsing fever (RF), patients experience shaking chills, a rise in temperature, and intensification of skin rashes known as the Jarisch-Herxheimerreaction (JHR). Experimental evidence by the present inventor indicates it is caused by nonendotoxinpyrogen and spirochetal lipoproteins. Mediation of the HerxheimerReaction in RF by the pro-inflammatory cytokines tumor necrosis factor (TNF), interleukin-6 (IL)-6, and interleukin-8 (IL-8) has been proposed.

Essential oils may reduce inflammatory cytokines in lab and animal studies. Herbal clinical results in Chinese hospitals are showing cytokine reduction and blood detoxification, and compounds which may block COVID-19 replication and cell penetration based on molecular modelling and lab studies on sars and COVID-19. Spearmint 115 mg/day adult dose (significantly reduced TNF-alpha, IL-1beta, and the expression of MMP-9, enhances Nrf2 protein expression). Eucalyptus globulus 600 mg/day adult dose (reduced inflammatory IL-1β, IL-4, IL-5, IL-6, IL-8, and TNF-α)

Turmeric 500 mg/day adult dose (Reduced MMP-2, MMP-9, cluster of differentiation 45 (CD45), TNF-α, interferon-γ (IFN-γ), IL-1β, IL-6, COX-2. Turmeric increased superoxide dismutase (SOD), glutathione, and glutathione reductase enzyme levels in blood and glutathione-S-transferase in the liver.

A liposome is a spherical vesicle having at least one lipid bilayer, is usually made with a phospholipid. Liposomes can be used as a vehicle for administration of essential oils or drugs to a subject. Liposomal essential oils may be able to target recurring symptoms, systemic infections, toxicity, inflammation in hard to reach reservoirs: cartilage, blood cells, collagen, nervous system, under biofilms. Liposomes form a barrier around their contents, which is resistant to enzymes in the mouth and stomach, alkaline solutions, digestive juices, bile salts, and intestinal flora that are generated in the human body, as well as free radicals. The contents of the liposomes are protected from oxidation and degradation. Liposomal oils can interact with several cell types including infected cells. This could also be a promising treatment for microbial infections.

Microparticle essential oils have antimicrobial properties. The small size of essential oil particles has a high surface tension. They fuse with and subsequently disrupt the membrane of isolated prokaryotic cells, viruses, and eukaryotic cells of fungi. They also do not affect eukaryotic cells of higher organisms.

Liposomes penetrate deeper into blood brain barrier, inside of cells, i.e. pathogen reservoirs.

As a natural and safe spice, the cinnamon oil exhibited a satisfactory antibacterial performance on MRSA and its biofilms. The application of liposomes further improves the stability of antimicrobial agents and extends the action time.

The present inventor has shown that encapsulating silver (as the ion Ag+) and tea tree oil (singly and in combination) in a controlled release liposomal carrier system can improve their antimicrobial efficacy as well as reduce the effective concentration required. These findings may impact on the problems of agent toxicity caused by the need for high effective doses or microbial resistance where long term application is required.

The combination of EO with fluoroquinolones, doxycycline, lincomycin, and maquindoxflorfenicol to treat ESBL-producing E. coli infections may lower, to a great extent, the effective dose of these antibiotics and thus minimize the side effects of antibiotics.

Thyme and peppermint EOs, and their active components and ciprofloxacin inhibit/eradicate biofilms of K. pneumoniae. There is a pronounced synergistic relationship between piperacillin/cinnamon bark oil, piperacillin/lavender oil, piperacillin/peppermint oil as well as meropenem/peppermint oil against two MDR bacteria strains. Amoxicillin antibacterial activity can be enhanced using active constituents present in oregano against the Methicillin-resistant Staphylococcus aureus infections.

A synergistic interaction was observed against all fifteen different strains of Gram positive and Gram-negative bacteria with the associations between the essential oils Pelargonium graveolens (Geranium Egypt)/Gentamicin. Myrtle oil showed significant antifungal activity when combined with amphotericin B against Candida albicans, different species of Aspergillus (A. niger, A. parasiticus, Aspergillus flavus). Lavender oil has the potential to reverse bacterial resistance to piperacillin in E. coli. Antibiotics+Essential Oils Combined. Basil essential oil combined with imipenem may increase antibacterial activity against S. aureus, and basil oil combined with ciprofloxacin worked synergistically against P. aeruginosa. Peppermint oil combined with gentamicin showed synergistic activity against Klebsiella pneumoniae. Caraway essential oil demonstrated synergy with gentamicin against K. pneumoniae. An additive effect was observed for gentamicin combined with thyme, fennel, basil, and clary sage against K. pneumoniae.

Tea tree oil, coriander and apple mint essential oils when used in combination with gentamicin and fluconazole showed a synergic effect on some bacteria and fungi. Caraway essential oil in combination with vancomycin demonstrated a potent synergistic effect against S. aureus.

An unexpectedly advantageous synergistic effect was found of four components of essential oils (thymol, eugenol, berberine and cinnamaldehyde) combined with streptomycin on planktonic and biofilm-associated food-borne pathogens Listeria monocytogenes and Salmonella typhimurium. Rosemary essential oil/ciprofloxacin displayed a favourable synergistic effect against K. pneumoniae. This patient was referred to a medical provider who administered intravenous silver, artesenuate, NAD+.

Improved with neurofeedback, Feeling 90% better. Symptoms are all gone except for occasional waking up in the middle of the night. Patient had multiple symptoms possibly due to a chronic herpes infection he may have gotten from a relationship. He tried antibiotics and supplements which did not help his symptoms. The inventor contracted with another company to provide an experimental scan to detect frequencies of underlying infections. HHV6 was one of the infections detected. The inventor provided customized microparticle remedies to the patient. He felt better when taking the remedies. He had lingering symptoms of fatigue and mental uneasiness. He was referred to another practitioner for IV treatments.

Referred to intravenous therapies (silver, artesenuate, NAD) ⋅Improved with neurofeedback. ⋅Feeling 90% better. —Symptoms are all gone except for occasional waking up in the middle of the night.

There are many challenges to diagnosing and helping people to heal chronic Lyme disease and multiple infections. It is a matter of finding the real underlying reasons and effective remedies and treatments—the most effective formulation for each patient may not be found immediately. It may take several tries. In order to target underlying infections, customized essential oil remedies give you a greater chance of success

In view of the present disclosure, the inventor believes that one skilled in the art would be able to determine suitable compositions, based on the present-indicated factors.

According to non-limiting example embodiments, the therapy or composition may be a combination formulation with more than one active ingredient, such as essential oils, herbs and/or supplements, or a therapy or composition to try to achieve more than one result. In any such combination therapy, the respective manufacturing processes and chemical environments would have to be compatible with one another. Example compositions may be quite useful in being able to provide a combination therapy composition for the treatment of at least two different infections or diseases in a single dosage form.

Also included herein are kits and/or systems that include one or more of the present microparticle compositions provided herein or one or more components of the compositions. The kits or systems may further include one or more of the following additional components, items, or other ingredients typically present in composition kits: instructions for use, administration implements or devices (such as vials, disinfectant wipes, etc.), disposable implements, additional treatment literature, additional implements or compositions for the treatment of various conditions treated by administration of the present substances of interest, such as e.g., anti-inflammatories, etc. In example embodiments, kits may include optionally one or more additional components to aid in the delivery of the microparticle composition to a subject in need thereof and/or instructions for administration.

The following are non-limiting example embodiment of compositions/formulations that may be used in accordance with the present invention and methods of their use.

### EXAMPLES

### Example 1

Based on case studies with patients, provided is a non-limiting example combination composition of microparticle essential oils that were effective in patients diagnosed with Bartonella:

Carrot seed EO 4%, celery seed EO 8%, cinnamon bark 1%, clove bud EO 1%, German chamomile EO 8%, copaiba EO 8%, geranium EO 4%, helichrysum EO 4%, lemongrass EO 13%, mountain savory EO 4%, oregano EO 5% spearmint EO 4%, and organic sunflower lecithin 36%, and reverse osmosis water was used. Other types of purified water may be used.

In this example, the essential oils were formed into microparticles using the sonication method.

### Example 2

Based on other case studies, provided is a non-limiting example combination composition of microparticle essential oils, which composition was effective in patients diagnosed with Borrelia and Babesia infections:

Roman chamomile EO 8%, cardamom seed EO 2%, cinnamon EO 1%, helichrysum EO 8%, lavender EO 8%, lemon balm 4%, thyme EO 12%, organic sunflower lecithin 37%, and reverse osmosis water.

In this example, the essential oils were formed into microparticles using the sonication method.

### Example 3

Based on other patient case studies, provided is a non-limiting example of a combination composition of microparticle essential oils that was effective in patients diagnosed with Borrelia infection:

Bergamot EO 2.5%, carrot seed EO 2.5%, Roman chamomile EO 5%, hyssop EO 2.5%, lemongrass EO 5%, marjoram EO 10%, nutmeg EO 5%, petitgrain EO 2.5%, thyme EO 10%, organic sunflower lecithin 65%, reverse osmosis water.

In this example, the essential oils were formed into microparticles using the sonication method.

### Example 4

Based on research studies, provided are non-limiting examples of combinations of essential oils that may be effective in patients diagnosed with, or suspected of having, a Covid-19 infection:

Bay Laurel EO 20%, Eucalyptus globulus EO 15%, organic sunflower lecithin 65%, reverse osmosis water.

Herba houttuyniae 8%, Ganoderma 8%, Folium isatidis 4%, Radix scutellariae 4%, Galla chinensis 4%, Radix astragali 4%, Radix et Rhizoma glycyrrhizae 3%

Other herbs with anti-Covid-19 properties which may be included in the above or other formulations used to treat patients with a Covid-19 infection include Folium mori, Flos chrysanthemi, Flos lonicerae japonica, Fructus forsythia, and/or Herba moslae, Rhizoma zingiberis, and Folium eriobotryae.

These herbs would be processed using one of the microencapsulation methods described above into microparticles which may be encapsulated in phosphatidylcholine from organic sunflower lecithin 65% and reverse osmosis water and may also be used for the treatment of Covid-19.

Although the present disclosure has been described in example embodiments, additional modifications and variations would be apparent to those skilled in the art. It is therefore to be understood that the present disclosure herein may be practiced other than as specifically described. Thus, the present embodiments should be considered in all respects as illustrative and not restrictive. Accordingly, it is intended that such changes and modifications fall within the scope of the present disclosure.

I claim:

1. A microparticle composition comprising:
one or more ingredients selected from essential oils, herbs, and supplements, that have been modified or synthesized to be in microparticle form;
wherein said one or more essential oils, herbs, and supplements in microparticle form are formulated to be pharmaceutically acceptable for administration in an effective amount to a subject, for treatment of an infection or disease in the subject, wherein the infection or disease is a Lyme tick borne illness related infection or disease, or a coronavirus.

2. The microparticle composition of claim 1, wherein the Lyme tick borne illness related infection or disease comprises one or more of infection or disease selected from the group consisting of Borrelia, Bartonella, Babesia, Ehrlichia, Anaplasmosis, Rickettsia, Mycoplasma, viruses, parasites, fungi, bacteria, and other tick infection.

3. The microparticle composition of claim 2, wherein the composition comprises microparticles of one or more of the following ingredients, Carrot seed essential oil (EO), celery seed EO, cinnamon bark, clove bud EO, German chamomile EO, copaiba EO, geranium EO, helichrysum EO, lemongrass EO, mountain savory EO, oregano EO, spearmint EO, organic sunflower lecithin, Roman chamomile EO, cardamom seed EO, cinnamon EO, lavender EO, lemon balm, thyme EO, organic sunflower lecithin, Bergamot EO, hyssop EO, marjoram EO, nutmeg EO, petitgrain EO, and thyme EO.

4. The microparticle composition of claim 3, further comprising purified water.

5. The microparticle composition of claim 1, wherein the coronavirus is Covid-19.

6. The microparticle composition of claim 5, wherein the composition comprises microparticles of one or more of the following ingredients Bay Laurel EO, Eucalyptus globulus EO, organic sunflower lecithin, Herba houttuyniae, Ganoderma, Folium isatidis, Radix scutellariae, Galla chinensis, Radix astragali, Radix et Rhizoma glycyrrhizae, Folium mori, Flos chrysanthemi, Flos lonicerae japonica, Fructus forsythia, Herba moslae, Rhizoma zingiberis, and Folium eriobotryae.

7. The microparticle composition of claim 6, further comprising purified water.

8. The microparticle composition of claim 1, wherein the one or more essential oils, herbs, and supplements, are modified or synthesized into one or more microparticle forms selected from the group consisting of liposomes, nanoparticles, nanostructured lipid carriers, and nano-emulsion compositions.

9. A method of making the microparticle composition of claim 1 comprising:
combining the one or more of the essential oils, herbs, and supplements; and modifying the essential oils, herbs, and supplements into microparticle form.

10. The method of claim 9, wherein the one or more essential oils, herbs, and supplements are modified into one or more microparticle forms selected from the group consisting of liposomes, nanoparticles, nanostructured lipid carriers, and nano-emulsion compositions.

11. The method of claim 9, wherein the one or more essential oils, herbs, and supplements are modified into microparticle form using sonication.

12. A method of treating a subject having a Lyme tick borne illness related infection or disease, or a coronavirus infection, comprising
administering to the subject the microparticle composition of claim 1 comprising one or more ingredients selected from essential oils, herbs, and supplements, that have been modified or synthesized to be in microparticle form,
in an effective amount to treat the Lyme tick borne illness related infection or disease, or a coronavirus infection.

13. The method of claim 12, wherein the subject is a mammal.

14. The method of claim 12, wherein the microparticle composition is administered to the subject by a method selected from the group consisting of orally, nasally, rectally, vaginally, topically, intravenously and injection.

15. The method of claim 12, wherein the Lyme tick borne illness related infection or disease comprises one or more of infection or disease selected from the group consisting of Borrelia, Bartonella, Babesia, Ehrlichia, Anaplasmosis, Rickettsia, Mycoplasma, viruses, parasites, fungi, bacteria, and other tick infection.

16. The method of claim 15, wherein the composition comprises microparticles of one or more of the following ingredients, Carrot seed EO, celery seed EO, cinnamon bark, clove bud EO, German chamomile EO, copaiba EO, geranium EO, helichrysum EO, lemongrass EO, mountain savory EO, oregano EO, spearmint EO, organic sunflower lecithin, Roman chamomile EO, cardamom seed EO, cinnamon EO, lavender EO, lemon balm, thyme EO, organic sunflower lecithin, Bergamot EO, hyssop EO, marjoram EO, nutmeg EO, petitgrain EO, and thyme EO.

17. The method of claim 12, wherein the coronavirus comprises COVID-19.

18. The method of claim 17, wherein the wherein the composition comprises microparticles of one or more of the following ingredients Bay Laurel EO, Eucalyptus globulus EO, organic sunflower lecithin, reverse osmosis water, Herba houttuyniae, Ganoderma, Folium isatidis, Radix scutellariae, Galla chinensis, Radix astragali, Radix et Rhizoma glycyrrhizae, Folium mori, Flos chrysanthemi, Flos lonicerae japonica, Fructus forsythia, Herba moslae, Rhizoma zingiberis, and Folium eriobotryae.

19. The method of claim 12, wherein the one or more essential oils, herbs, and supplements; are modified into one or more microparticle forms selected from the group consisting of liposomes, nanoparticles, nanostructured lipid carriers, and nano-emulsion compositions.

20. A kit comprising one or more microparticle compositions according to claim 1 or a component or ingredient thereof and optionally one or more additional components to aid in the delivery of the microparticle composition to a subject in need thereof and/or instructions for administration.
